(19) United States
(12) Patent Application Publication
(10) Pub. No.: US 2025/0299792 A1
(43) Pub. Date: Sep. 25, 2025

(54) INFORMATION PROCESSING DEVICE AND INFORMATION PROCESSING METHOD

(71) Applicant: Hitachi, Ltd., Tokyo (JP)

(72) Inventors: Kunihiko KIDO, Tokyo (JP); Yoichi Nakamoto, Tokyo (JP)

(73) Assignee: Hitachi, Ltd., Tokyo (JP)

(21) Appl. No.: 19/015,484

(22) Filed: Jan. 9, 2025

(30) Foreign Application Priority Data
Mar. 21, 2024 (JP) 2024-044776

(51) Int. Cl. G16H 20/10; G16H 10/60

(52) U.S. Cl. CPC G16H 20/10; G16H 10/60

(57) ABSTRACT

An information processing device holds: a life cycle model indicating information for estimating a dynamic change of states of a biological system or an ecological system into which the target product is introduced, and the target product and a used item other than the target product used in each state; environmental load information indicating an environmental load of each of the target product and the used item; and index information indicating an index relating to, for each state, a quality of life of the biological system or the ecological system affected by the target product, and estimates the dynamic change of the states based on the life cycle model; calculates the environmental load and the index in an entire period of the estimated dynamic change with reference to the environmental load information and the index information; and corrects the calculated environmental load based on the calculated index.

### REFERENCE TO RELATED APPLICATION

The present application claims priority from Japanese patent application JP 2024-044776 filed on Mar. 21, 2024, the content of which is hereby incorporated by reference into this application.

### BACKGROUND OF THE INVENTION

The present invention relates to an information processing device and an information processing method.

As background art in this technical field, there exists JP 2011-204217 A. In 2011-204217 A, it is described that “an environmental load simulation device for simulating an environmental load of a target product includes a screen generation module which generates a screen for a user to input a setting condition for a preset process included in a life cycle, a greenhouse gas emission amount calculation module which refers to a database storing relevant information for calculating a greenhouse gas emission amount in advance based on the setting condition input to the screen generated by the screen generation module and calculates the greenhouse gas emission amount from the relevant information corresponding to the setting condition which is acquired from the database, and a simulation module which simulates the environmental load of the target product by using the calculation result” (see Abstract).

In the technology as described in JP 2011-204217 A, products constructed of many types of raw materials are centrally managed, and the environmental load of the products is simulated based on the greenhouse gas emission amount generated over the life cycle of the product. However, when the target product is a product that is introduced into a biological system or an ecological system, such as a pharmaceutical product, the introduction of the product into the biological system or the ecological system may affect the state of the biological system or the ecological system. This may cause the state of the biological system or the ecological system to dynamically change, and as a result, cause the environmental load to change. The technology as described in JP 2011-204217 A does not take such a dynamic change into consideration in calculating the environmental load.

### SUMMARY OF THE INVENTION

Thus, at least one aspect of this invention is to appropriately evaluate an environmental load relating to an evaluation target product which is introduced into a biological system or an ecological system.

The at least one aspect of this invention adopts the following structures in order to solve the above problems. An information processing device comprises: a processor; and a memory, the memory holds: a life cycle model indicating information for estimating a dynamic change of states relating to an evaluation target product of one of a biological system or an ecological system into which the evaluation target product is introduced, and the evaluation target product and a used item other than the evaluation target product which are used in each of the states; environmental load information indicating an environmental load in a production process and a distribution process of each of the evaluation target product and the used item; and index information indicating an index value relating to, for each of the states, a quality of life of the one of the biological system or the ecological system affected by the evaluation target product, and the processor is configured to: estimate the dynamic change of the states based on the life cycle model and identify the evaluation target product and the used item used in each of the states which stays in the estimated dynamic change; calculate the environmental load in an entire period of the estimated dynamic change based on the environmental load corresponding to the identified evaluation target product and used item in the environmental load information; calculate the index value in the entire period of the estimated dynamic change based on the index value corresponding to each of the states which stays in the estimated dynamic change in the index information; correct the calculated environmental load based on the calculated index value; and generate data for displaying the corrected environmental load.

The at least one aspect of this invention can appropriately evaluate an environmental load relating to an evaluation target product which is introduced into a biological system or an ecological system.

Problems, configurations, and effects which are not mentioned above are explained in the following embodiments.

### BRIEF DESCRIPTION OF THE DRAWINGS

FIG. 1 is a block diagram for illustrating a function configuration example of a life cycle assessment processing device according to the First Embodiment.

FIG. 2 is a block diagram for illustrating a hardware configuration example of the life cycle assessment processing device according to the First Embodiment.

FIG. 3 is an explanatory diagram for illustrating an example of processing executed by an emission amount calculation module and an emission amount correction module according to the First Embodiment.

FIG. 4 is a table for showing a data configuration example of QALY (Quality-Adjusted Life Years) data according to the First Embodiment.

FIG. 5A is a table for showing a data configuration example of pharmaceutical product inventory data according to the First Embodiment.

FIG. 5B is a table for showing a data configuration example of medical material inventory data according to the First Embodiment.

FIG. 5C is a table for showing a data configuration example of metabolite inventory data according to the First Embodiment.

FIG. 6 is an explanatory diagram for illustrating an example of a clinical condition transition model according to the First Embodiment.

FIG. 7 is a flowchart for illustrating an example of life cycle assessment processing according to the First Embodiment.

FIG. 8 is a diagram for illustrating a screen configuration example of an assessment processing result display screen according to the First Embodiment.

FIG. 9 is a diagram for illustrating another screen configuration example of the assessment processing result display screen according to the First Embodiment.

FIG. 10 is an explanatory diagram for illustrating an example of processing executed by the emission amount calculation module and the emission amount correction module according to the Second Embodiment.

FIG. 11 is a table for showing a data configuration example of evaluation index data according to the Second Embodiment.

FIG. 12A is a table for showing a data configuration example of material inventory data according to the Second Embodiment.

FIG. 12B is a table for showing a data configuration example of waste product inventory data according to the Second Embodiment.

FIG. 13 is an explanatory diagram for illustrating an example of an ecological system model according to the Second Embodiment.

FIG. 14 is a diagram for illustrating a screen configuration example of the assessment processing result display screen according to the Second Embodiment.

### DETAILED DESCRIPTION OF THE PREFERRED EMBODIMENTS

In the following, an embodiment of the present invention is explained referring the attached drawings. The embodiment is an example to achieve the present invention and does not limit a technical range of the present invention. In the drawings, the same configuration has the same reference letter.

### First Embodiment

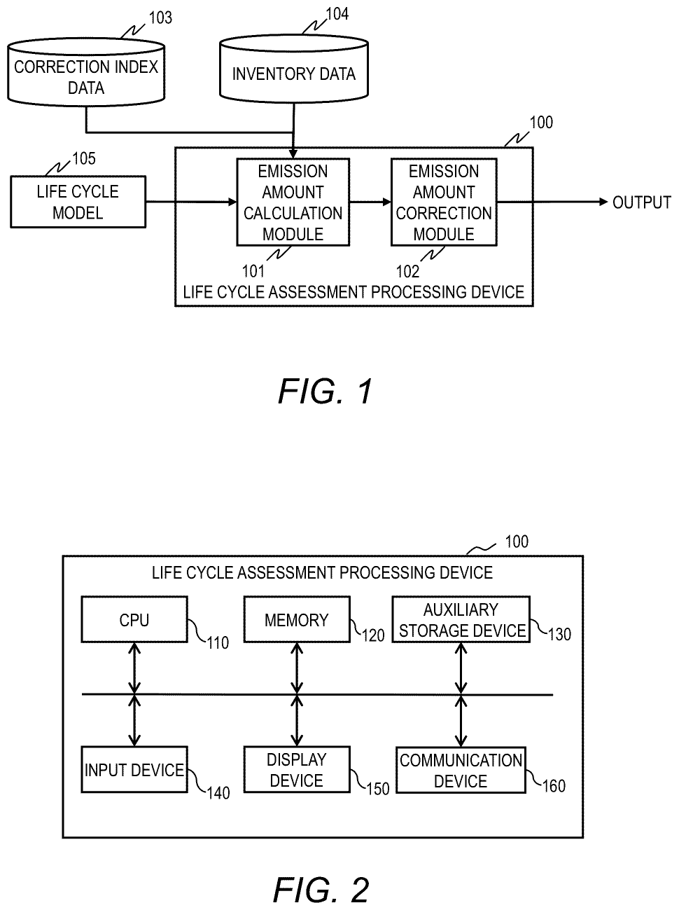

FIG. 1 is a block diagram for illustrating a function configuration example of a life cycle assessment processing device. A life cycle assessment processing device 100 evaluates an emission amount relating to an evaluation target product. The evaluation target product in a first embodiment of this invention is, for example, introduced into (or used in) a biological system or an ecological system. In the first embodiment, pharmaceutical products (including, for example, low-molecular-weight pharmaceutical products and biopharmaceutical products, which are biological products) that are introduced into living organisms (for example, the human body) and bioplastics (biological products) that are introduced into soil, seawater, and the like during degradation are both examples of evaluation target products.

The life cycle assessment processing device 100 includes, for example, an emission amount calculation module 101 and an emission amount correction module 102, both of which are functional modules. The emission amount calculation module 101 acquires correction index data 103, inventory data 104, and a life cycle model 105. The life cycle assessment processing device 100 may hold the correction index data 103, the inventory data 104, and the life cycle model 105 in advance.

The life cycle model 105 is a model for estimating a dynamic change (life cycle) of states relating to an evaluation target product of a biological system or an ecological system into which the evaluation target product is introduced (in which the evaluation target product is used). The life cycle model 105 indicates, for example, a plurality of stages included in a life cycle and transition probabilities between stages. As the life cycle model 105, for example, a Markov chain model is used.

The inventory data 104 indicates a greenhouse gas (GHG) emission amount in the production process and/or distribution process of the evaluation target product, and the GHG emission amount relating to the use of the evaluation target product. The GHG emission amount is an example of an environmental load, and the inventory data 104 is an example of environmental load information. The GHG emission amount is hereinafter also simply referred to as “emission amount.”

The correction index data 103 is an index for correcting the emission amount, the index relating to a quality of life of the biological system or the ecological system affected by the introduction of the evaluation target product. For example, a quality-adjusted life year (QALY) of a person into which a pharmaceutical product is introduced is an example of this index.

The emission amount calculation module 101 calculates an emission amount which takes into account changes in the stages of the biological system or the ecological system based on the inventory data 104 and the life cycle model 105. The emission amount calculation module 101 calculates an index value relating to the quality of life of the biological system or the ecological system which takes into account changes in the stages of the biological system or the ecological system based on the correction index data 103 and the life cycle model 105.

The emission amount correction module 102 uses the index value calculated by the emission amount calculation module 101 to correct the emission amount calculated by the emission amount calculation module 101, and outputs the corrected emission amount.

FIG. 2 is a block diagram for illustrating a hardware configuration example of the life cycle assessment processing device 100. The life cycle assessment processing device 100 is formed of, for example, a computer including a central processing unit (CPU) 110, a memory 120, an auxiliary storage device 130, an input device 140, a display device 150, and a communication device 160.

The CPU 110 is an example of a processor, and executes a program stored in the memory 120. The memory 120 includes a read only memory (ROM), which is a nonvolatile memory device, and a random access memory (RAM), which is a volatile memory device. The ROM stores, for example, an invariant program (for example, basic input/output system (BIOS)). The RAM is a dynamic random access memory (DRAM) or other such high-speed and volatile memory device, and temporarily stores a program to be executed by the CPU 110 and data to be used when the program is executed.

The auxiliary storage device 130 is, for example, a large-capacity and non-volatile storage device, such as a magnetic storage device (hard disk drive (HDD)) and a flash memory (solid state drive (SSD)). Programs to be executed by the CPU 110 and data to be used when the programs are executed are stored in the auxiliary storage device 130. Specifically, the programs are read out from the auxiliary storage device 130, loaded onto the memory 120, and executed by the CPU 110.

The input device 140 is a device, such as a keyboard or a mouse, through which input from an operator is received. The display device 150 is a device, such as a display or a printer, which outputs an execution result of a program in a form which the operator can visually recognize.

The communication device 160 is a network interface device which controls communication to and from another device in accordance with a predetermined protocol. The communication device 160 may include, for example, a serial interface such as a universal serial bus (USB).

A part or all of programs executed by the CPU 110 may be supplied, to the life cycle assessment processing device 100, from a removable medium (such as a CD-ROM or a flash memory) being a non-transitory storage medium or from an external computer provided with a non-transitory storage device via a network, and may be stored in the nonvolatile auxiliary storage device 130 being a non-transitory storage medium. Thus, it is preferred that life cycle assessment processing device 100 include an interface for reading data from the removable medium.

The life cycle assessment processing device 100 is a computer system formed on physically one computer or formed on a plurality of computers that are configured logically or physically, and may be operated on separate threads on the same computer, or may operate on a virtual machine built on a plurality of physical computer resources.

The CPU 110 includes, for example, the above-mentioned functional module, that is, the emission amount calculation module 101 and the emission amount correction module 102. For example, the CPU 110 functions as the emission amount calculation module 101 by operating in accordance with an emission amount calculation program loaded onto the memory 120, and functions as the emission amount correction module 102 by operating in accordance with an emission amount correction program loaded onto the memory 120.

A part or all of the functions implemented by the functional modules included in the CPU 110 may be implemented by hardware, for example, an application specific integrated circuit (ASIC), a field-programmable gate array (FPGA), or the like.

The auxiliary storage device 130 stores, for example, the correction index data 103, the inventory data 104, and the life cycle model 105 acquired by the life cycle assessment processing device 100. In the first embodiment, a part or all of the information stored in the auxiliary storage device 130 may be stored in the memory 120, or may be stored in an external database, for example, coupled to the life cycle assessment processing device 100.

In the embodiments, information used by the life cycle assessment processing device 100 does not depend on its data structure, and may be expressed in any data structure. For example, a data structure appropriately selected from a table, a list, a database, and a queue can store the information.

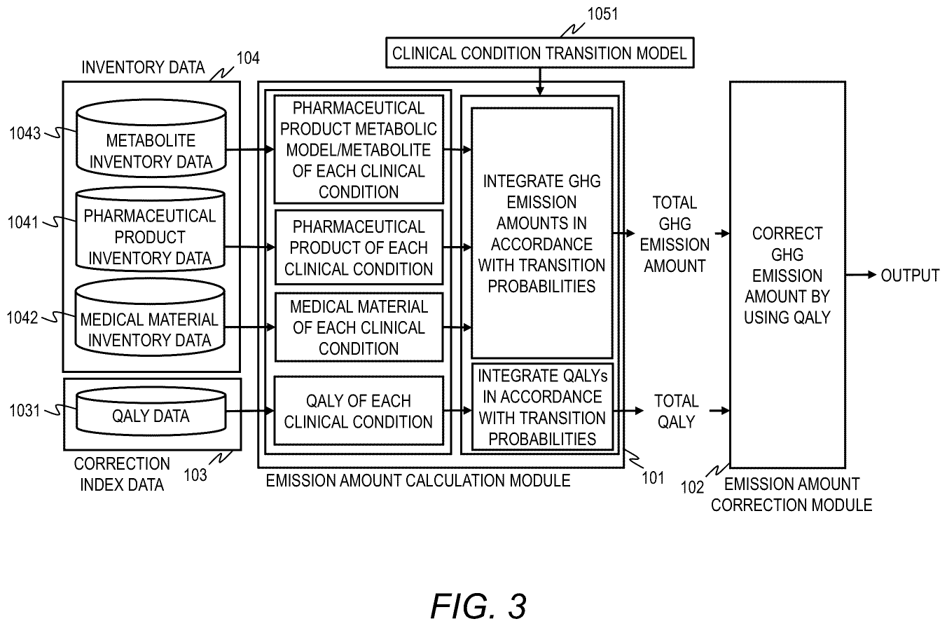

FIG. 3 is an explanatory diagram for illustrating an example of processing executed by the emission amount calculation module 101 and the emission amount correction module 102. In the first embodiment, when an evaluation-target pharmaceutical product (evaluation target product) is administered to a patient (an example of a biological system) who is suffering from an indication or who wishes to prevent the indication, the life cycle assessment processing device 100 calculates the emission amount relating to the evaluation-target pharmaceutical product by taking into account the life cycle of the patient.

The emission amount calculation module 101 acquires, as the life cycle model 105, a clinical condition transition model 1051 corresponding to a combination of the evaluation-target pharmaceutical product and an indication. Specifically, for example, the clinical condition transition model 1051 indicates a plurality of clinical conditions (a clinical condition is an example of a stage in the life cycle of the patient) under which a patient may fall in the indication, and transition probabilities between the clinical conditions. Further, the clinical condition transition model 1051 indicates the pharmaceutical products and medical materials used in each clinical condition of the indication. The medical materials may or may not be a biological product.

In at least one clinical condition of the clinical condition transition model 1051, the evaluation-target pharmaceutical product corresponding to the clinical condition transition model 1051 is used. In other words, the impact on the state of the patient from the introduction of the evaluation-target pharmaceutical product into the patient (living organism) is reflected in the clinical condition transition model 1051. Further, for each clinical condition of the clinical condition transition model 1051, pharmaceutical products and/or medical materials different from the evaluation-target pharmaceutical product may be used.

The clinical condition transition model 1051 can be said to be a model for estimating a dynamic change of the states (the clinical conditions of the indication of an evaluation-target pharmaceutical product, which is an example of the evaluation target product) relating to the evaluation-target pharmaceutical product of a patient (living organism) into which the evaluation-target pharmaceutical product is introduced.

The inventory data 104 includes, for example, pharmaceutical product inventory data 1041, medical material inventory data 1042, and metabolite inventory data 1043. The pharmaceutical product inventory data 1041 indicates the emission amount in the production process and/or distribution process of the pharmaceutical product used in the clinical conditions included in the clinical condition transition model 1051.

The medical material inventory data 1042 indicates the emission amount in the production process and/or distribution process of the medical materials used in the clinical conditions included in the clinical condition transition model 1051. The metabolite inventory data 1043 indicates a metabolic model indicating a metabolite produced when the pharmaceutical product indicated by the pharmaceutical product inventory data 1041 is introduced into the body of a patient, and the emission amount produced by the production of the metabolite.

The correction index data 103 includes QALY data 1031. The QALY data 1031 indicates the QALY of a patient for each clinical condition included in the clinical condition transition model 1051.

The emission amount calculation module 101 identifies, for each clinical condition included in the clinical condition transition model 1051, the emission amount corresponding to the pharmaceutical product, the emission amount corresponding to the medical material, the emission amount corresponding to the metabolite corresponding to the pharmaceutical product, and the QALY from the pharmaceutical product inventory data 1041, the medical material inventory data 1042, the metabolite inventory data 1043, and the QALY data 1031, respectively.

The emission amount calculation module 101 causes the clinical condition to transition in accordance with the transition probabilities indicated by the clinical condition transition model 1051, and integrates the emission amounts and the QALYs for each clinical condition. The emission amount calculation module 101 outputs the integrated emission amount (total emission amount) and the integrated QALY (total QALY) to the emission amount correction module 102. The total emission amount indicates the total of the emission amounts for a product relating to a disease which takes into account the life cycle when the evaluation-target pharmaceutical product is introduced into a patient having any of the clinical conditions of an indication. The total QALY indicates the total of the QALYs of a patient which takes into account the life cycle when the evaluation-target pharmaceutical product is introduced into a patient having any of the clinical conditions of an indication. The emission amount correction module 102 corrects the total emission amount by using the total QALY, and outputs the corrected emission amount.

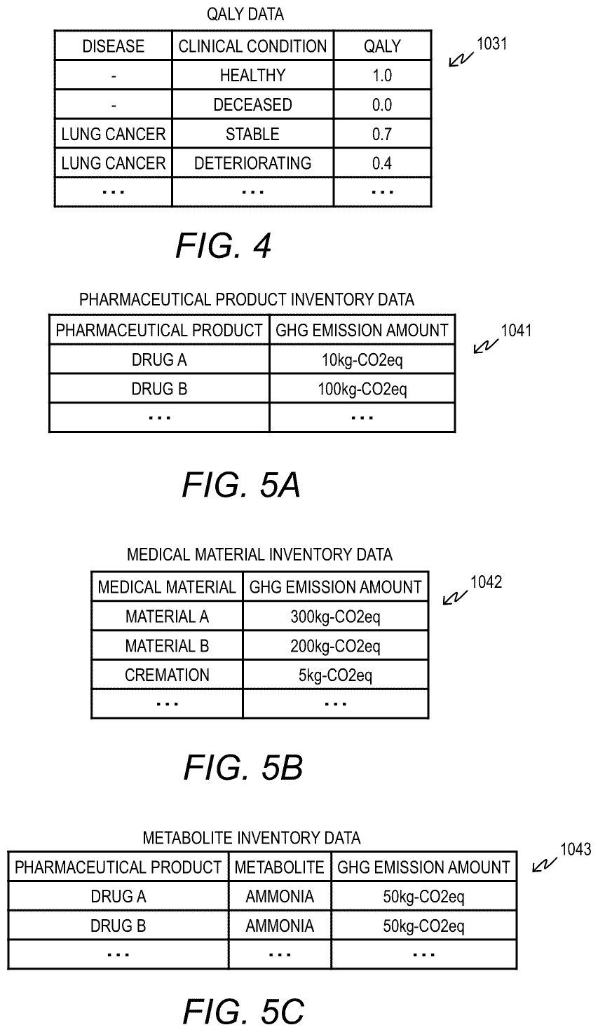

FIG. 4 is a table for showing a data configuration example of the QALY data 1031. The QALY data 1031 holds a QALY value for each combination of a disease and a clinical condition. It should be noted that the clinical condition “healthy” has no corresponding disease defined, and the QALY is 1.0. Moreover, the clinical condition “deceased” has no corresponding disease defined, and the QALY is 0.0. The QALY data 1031 is not required to include the disease field. That is, when the clinical condition is the same regardless of the disease, the QALY values may also be the same. In the first embodiment, QALY is defined as a value of 0.0 or more (1.0 or less) by using, for example, EuroQol 5 Dimension (EQ-5D).

FIG. 5A is a table for showing a data configuration example of the pharmaceutical product inventory data 1041. The pharmaceutical product inventory data 1041 indicates the emission amount (emission amount in the production process and/or distribution process of the pharmaceutical product) corresponding to each predetermined unit amount (for example, one tablet) of the pharmaceutical product.

FIG. 5B is a table for showing a data configuration example of the medical material inventory data 1042. The medical material inventory data 1042 indicates the emission amount (emission amount in the production process and/or distribution process of the medical material) corresponding to each predetermined unit amount (for example, one) of the medical material. In the example of FIG. 5B, the emission amount relating to cremation, which is performed when the clinical condition of the patient transitions to “deceased,” is defined in the medical material inventory data 1042 for convenience, but the emission amount relating to cremation may be defined in another inventory.

FIG. 5C is a table for showing a data configuration example of the metabolite inventory data 1043. The metabolite inventory data 1043 indicates the metabolite (metabolic model) produced when a pharmaceutical product is introduced into the body of the patient, and the emission amount produced by the production of the metabolite when the predetermined unit amount of the pharmaceutical product is introduced into the body.

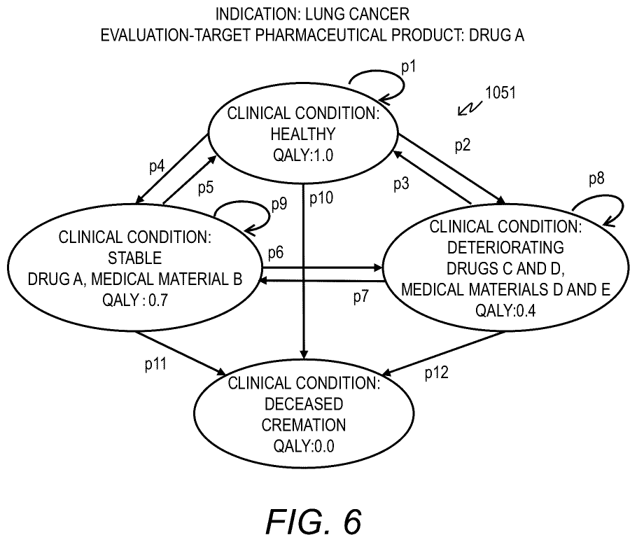

FIG. 6 is an explanatory diagram for illustrating an example of the clinical condition transition model 1051. In FIG. 6, there is illustrated an example of a clinical condition transition model 1051 (Markov chain model) in which the indication is “lung cancer” and the evaluation-target pharmaceutical product is “drug A.”

In the clinical condition transition model 1051, a plurality of clinical conditions (“healthy,” “stable,” “deteriorating,” and “deceased”) and the pharmaceutical product and/or medical material to be used in diagnosis and treatment for each clinical condition are defined. Like in the case in which the clinical condition is “healthy” in the example of FIG. 6, there may be a clinical condition in which no pharmaceutical products or medical materials are used.

In the example of FIG. 6, the QALY value of each clinical condition is also defined in the clinical condition transition model 1051. However, at the stage at which the emission amount calculation module 101 acquires the clinical condition transition model 1051, the QALY value is not defined, and the emission amount calculation module 101 assigns the QALY of each clinical condition to the clinical condition transition model 1051 based on the QALY data 1031 in Step S703, which is described later. The QALY value may also be defined in advance in the clinical condition transition model 1051 acquired by the emission amount calculation module 101. In this case, the emission amount calculation module 101 is not required to acquire the QALY data 1031, and the above-mentioned processing of assigning the QALY can also be omitted.

Each arrow in the clinical condition transition model 1051 indicates a transition between clinical conditions. In the clinical condition transition model 1051, there may be cases in which transitions to the same clinical condition are defined. In the example of FIG. 6, a transition from “healthy” to “healthy,” a transition from “stable” to “stable,” and a transition from “deteriorating” to “deteriorating” are defined.

Further, in the clinical condition transition model 1051, there may be cases in which transitions between a specific clinical condition are not defined. For example, when a patient is “deceased,” the clinical condition of the patient does not change after that, and thus in the example of FIG. 6, transitions from “deceased” to “deceased,” from “deceased” to “healthy,” from “deceased” to “stable,” and from “deceased” to “deteriorating” are not defined.

In the clinical condition transition model 1051, p1 to p12 attached to respective arrows each indicate the transition probability of the clinical condition of the patient transitioning to the clinical condition indicated by the end point of the arrow when a predetermined unit period (for example, one month) has elapsed since the clinical condition of the patient transitioned to the clinical condition indicated by the starting point of the arrow.

For example, for a certain one patient having a “stable” clinical condition, a unit amount (which is the same as the unit amount corresponding to the emission amount indicated by the pharmaceutical product inventory data 1041) of a drug A and a predetermined unit amount of a medical material B (which is the same as the unit amount corresponding to the emission amount indicated by the medical material inventory data 1042) are used for the predetermined unit period. Under those conditions, when the predetermined unit period has elapsed since the clinical condition of the patient transitioned to “stable,” the clinical condition of the patient transitions to “healthy” at a transition probability p5, transitions to “deteriorating” at a transition probability p6, transitions to “stable” at a transition probability p9, and transitions to “deceased” at a transition probability p11.

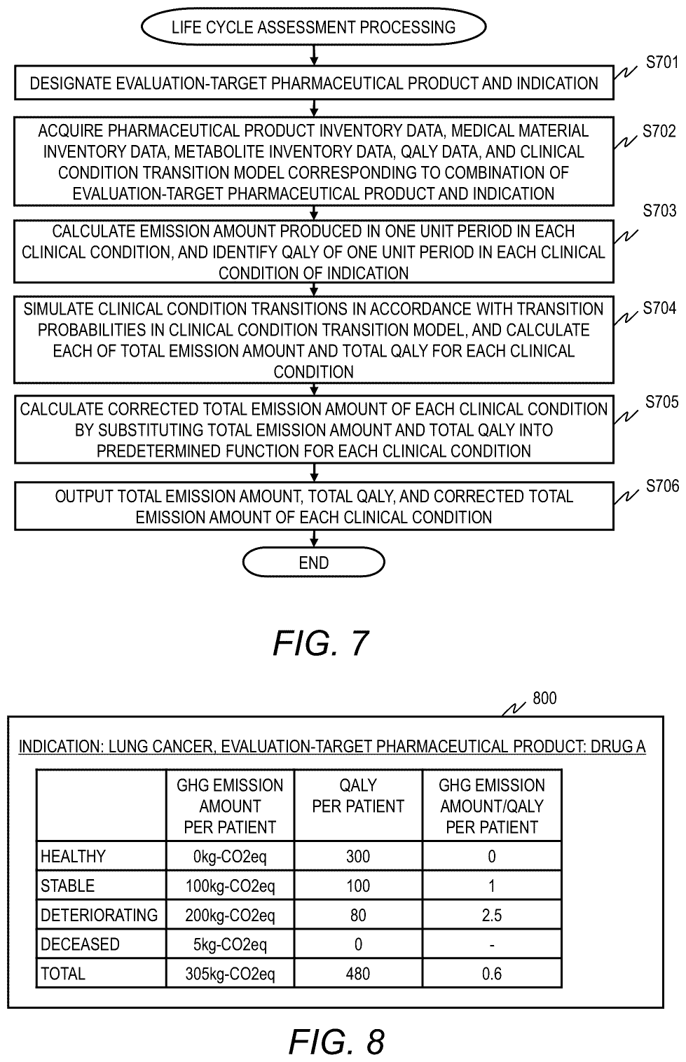

FIG. 7 is a flowchart for illustrating an example of life cycle assessment processing. The emission amount calculation module 101 receives a designation of the indication and the evaluation-target pharmaceutical product, for example, through input to the input device 140 (S701).

The emission amount calculation module 101 acquires the pharmaceutical product inventory data 1041, the medical material inventory data 1042, the metabolite inventory data 1043, the QALY data 1031, and the clinical condition transition model 1051 corresponding to the combination of the indication and the evaluation-target pharmaceutical product designated in Step S701, for example, by receiving those pieces of data and the clinical condition transition model 1051 from an external server or by receiving those pieces of data and the clinical condition transition model 1051 through input to the input device 140 (S702).

The emission amount calculation module 101 calculates the emission amount produced in one unit period in each clinical condition included in the clinical condition transition model 1051 acquired in Step S702, and identifies the QALY of the one unit period in each clinical condition of the relevant indication from the QALY data 1031 (S703).

Specifically, in Step S703, for example, the emission amount calculation module 101 identifies the pharmaceutical product and medical material to be used in each clinical condition included in the clinical condition transition model 1051. The emission amount calculation module 101 acquires the emission amount corresponding to each identified pharmaceutical product from the pharmaceutical product inventory data 1041, acquires the emission amount corresponding to each identified medical material from the medical material inventory data 1042, and acquires the emission amount corresponding to the metabolite produced from each identified pharmaceutical product from the metabolite inventory data 1043. The emission amount calculation module 101 calculates the emission amount produced in one unit period in each clinical condition by calculating, for each clinical condition, the total of the emission amount corresponding to each pharmaceutical product and medical material to be used, and the emission amount corresponding to the metabolite produced from each pharmaceutical product to be used.

The emission amount calculation module 101 calculates the total emission amount and the total QALY of each clinical condition in the entire simulation period by simulating the clinical condition transitions of the patient in accordance with the transition probabilities indicated by the clinical condition transition model 1051 and integrating the emission amounts and QALYs of each clinical condition (S704).

Specifically, for example, the emission amount calculation module 101 sets the clinical condition for one patient to an initial value (for example, “healthy”), and in accordance with the transition probabilities indicated by the clinical condition transition model 1051, simulates the state of the clinical condition of the patient for every unit period that elapses until the clinical condition of the patient transitions to “deceased.” When the clinical condition of the patient does not transition to “deceased” even after a predetermined number of unit periods have elapsed, the simulation may be terminated. The transitioning of the clinical condition of the patient to “deceased” and the elapse of a predetermined number of unit periods are both examples of simulation termination conditions.

The emission amount calculation module 101 integrates, for each clinical condition, the emission amount produced in one unit period in the clinical condition calculated in Step S703 together with the transition of the clinical condition of the patient in the simulation. Similarly, the emission amount calculation module 101 integrates, for each clinical condition, the QALY of one unit period in the clinical condition identified in Step S703 together with the transition of the clinical condition of the patient in the simulation.

The emission amount calculation module 101 may calculate, for example, the number of periods (period length) that the patient stays in each clinical condition in the entire simulation period, and calculate the emission amount of each clinical condition in the simulation period by multiplying, for each clinical condition, the calculated number of periods by the emission amount produced in one unit period in that clinical condition calculated in Step S704. Similarly, the emission amount calculation module 101 may calculate the emission amount of each clinical condition in the simulation period, for example, by multiplying, for each clinical condition, the calculated number of periods by the QALY in the clinical condition identified in Step S703.

In the example described above, the emission amount calculation module 101 executes the simulation for one patient, but the emission amount calculation module 101 may execute the simulation for a plurality of patients. In this case, the emission amount calculation module 101, for example, executes the above-mentioned simulation for each of the plurality of patients, and calculates the total of the emission amount of each clinical condition and the total of the QALY for each clinical condition. Further, the emission amount calculation module 101 calculates the total of the emission amount of each clinical condition for the plurality of patients as a whole by summing the emission amount of each patient for each clinical condition, and calculates the total of the QALY for each clinical condition for the plurality of patients as a whole by summing the QALY of each patient for each clinical condition.

The emission amount correction module 102 calculates the corrected emission amount of each clinical condition by substituting, for each clinical condition, the total emission amount calculated in Step S704 and the total QALY calculated in Step S704 into a predetermined function (S705). The predetermined function is defined such that the corrected total emission amount becomes larger as the (before-correction) total emission amount becomes larger, and the corrected total emission amount becomes smaller as the total QALY becomes larger. For example, a function which divides the emission amount by the QALY is an example of the predetermined function.

The emission amount correction module 102 outputs an assessment processing result display screen showing the total emission amount of each clinical condition calculated in Step S704, the total QALY of each clinical condition calculated in Step S704, and the corrected total emission amount of each clinical condition calculated in Step S705 to, for example, the display device 150 (S706), and ends the life cycle assessment processing.

In Step S706, the emission amount correction module 102 may display, in addition to or in place of the total emission amount, the total QALY, and the corrected total emission amount, an emission amount per person (a value obtained by dividing the total emission amount by the number of patients in the simulation), a QALY per person (a value obtained by dividing the total QALY by the number of patients in the simulation), and a corrected emission amount per person (a value obtained by dividing the corrected total emission amount by the number of patients in the simulation) on the assessment processing result display screen.

In Step S702 to Step S705 in the life cycle assessment processing described above, the emission amount calculation module 101 can calculate the emission amount relating to the evaluation-target pharmaceutical product with high precision by taking into account not only the emission amount corresponding to the evaluation-target pharmaceutical product, but also the emission amounts corresponding to pharmaceutical products other than the evaluation-target pharmaceutical product and medical materials used for diagnosis and treatment in each clinical condition indicated by the clinical condition transition model 1051 (pharmaceutical products other than the evaluation-target pharmaceutical product and medical materials used in each clinical condition are both examples of a “used item” used in each clinical condition), as well as the emission amounts corresponding to the metabolites of the evaluation-target pharmaceutical product and pharmaceutical products other than the evaluation-target pharmaceutical product used in each clinical condition (the metabolite produced by each clinical condition is an example of a “produced substance” in each clinical condition).

The emission amount calculation module 101 can calculate the emission amount over the entire estimated life cycle of the patient (entire period of dynamic change in states) with high precision by simulating the transitions of the clinical condition of the patient by using the clinical condition transition model 1051, and integrating the emission amounts in the simulated clinical conditions.

For example, it is assumed that, for two evaluation-target pharmaceutical products to be administered to “stable” patients, one of the two evaluation-target pharmaceutical products may have a large emission amount but the probability of the patient administered with that evaluation-target pharmaceutical product transitioning to “deteriorating” may be low, whereas the other of the two evaluation-target pharmaceutical products may have a small emission amount but the probability of the patient administered with that evaluation-target pharmaceutical product transitioning to “deteriorating” may be high. Further, it is assumed that the emission amount corresponding to the drug administered to the “deteriorating” patient is large. In such a case, when the emission amount is evaluated by focusing only on the evaluation-target pharmaceutical products, the emission amount corresponding to the other of the two evaluation-target pharmaceutical products is evaluated as having a small emission amount, but as described above, by using the clinical condition transition model 1051, the emission amount calculation module 101 can evaluate which of the evaluation-target pharmaceutical products to administer to “stable” patients has the smaller emission amount over the entire life cycle of the patient.

Further, in the evaluation of the emission amount of a product introduced into a living organism, such as a pharmaceutical product, when only the magnitude of the emission amount is evaluated, for example, a high evaluation may be given in cases in which the emission amount is smaller because the clinical condition of the patient has transitioned to “deceased” and hence medical materials and pharmaceutical products are no longer used by the patient, or cases in which the patient has died because the patient chose a pharmaceutical product having a lower therapeutic effect or stopped treatment (that is, cases in which the quality of life of the living organism into which the evaluation-target pharmaceutical product is introduced decreased). Therefore, in Step S705 described above, the emission amount correction module 102 corrects the emission amount so that the emission amount is reduced when the QALY indicating the quality of life of the patient is higher, and thus can evaluate both the emission amount and the quality of life of the patient.

In the above-mentioned example, the life cycle assessment processing device 100 calculates the emission amount, total QALY, and corrected emission amount for each clinical condition, but the life cycle assessment processing device 100 may calculate each of those values as only the total value for all clinical conditions.

FIG. 8 is a diagram for illustrating a screen configuration example of the assessment processing result display screen which is output in Step S706. An assessment processing result display screen 800 displays, for example, information indicating the indication (“lung cancer”) and the evaluation-target pharmaceutical product (“drug A”) designated in Step S701. Further, the assessment processing result display screen 800 displays information indicating the emission amount per patient, the QALY per patient, and the corrected emission amount (emission amount/QALY) per patient for each clinical condition as a total value.

The assessment processing result display screen 800 of FIG. 8 allows the user of the life cycle assessment processing device 100 to recognize the emission amount, the QALY, and the corrected emission amount for each clinical condition.

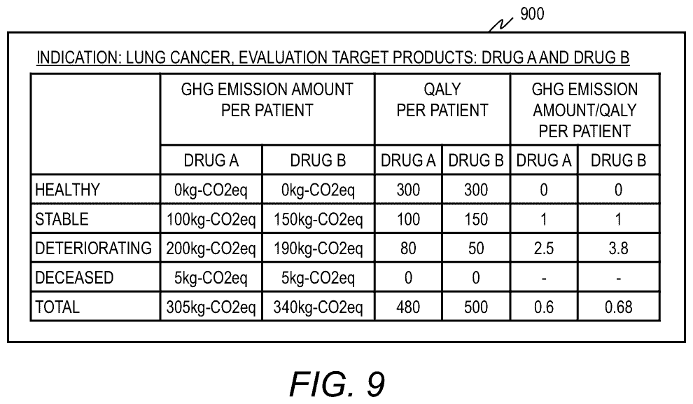

FIG. 9 is a diagram for illustrating another screen configuration example of the assessment processing result display screen which is output in Step S706. In Step S701, a plurality of evaluation-target pharmaceutical products (“drug A” and “drug B”) for the same indication (“lung cancer”) have been designated, and the processing steps of Step S702 to Step S705 have been executed on each of the combination of the indication and one of the evaluation-target pharmaceutical products (“drug A”) and the combination of the indication and another of the evaluation-target pharmaceutical products (“drug B”).

The assessment processing result display screen 900 displays, for example, information indicating the indication (“lung cancer”) and the evaluation-target pharmaceutical products (“drug A” and “drug B”) designated in Step S701. Further, the assessment processing result display screen 900 displays, for each evaluation-target pharmaceutical product, information indicating the emission amount per patient, the QALY per patient, and the corrected emission amount per patient (emission amount/QALY) for each clinical condition as a total value.

The assessment processing result display screen of FIG. 9 allows the user of the life cycle assessment processing device 100 to compare the emission amount, the QALY, and the corrected emission amount for each clinical condition for different evaluation-target pharmaceutical products. In particular, for example, it is possible to easily recognize situations in which the magnitude of the emission amounts are reversed when the QALY is taken into account, such as when the emission amount (before correction) of one evaluation-target pharmaceutical product is smaller than the emission amount (before correction) of another evaluation-target pharmaceutical product, but the corrected emission amount of the one evaluation-target pharmaceutical product is larger than the corrected emission amount of the another evaluation-target pharmaceutical product.

### Second Embodiment

In a second embodiment of this invention, an example is described in which the evaluation target product is a bioplastic product (biological product having biodegradability). For example, there is a life cycle in which a manufacturer in a certain region produces a bioplastic product (production stage), a consumer living in the region uses the produced bioplastic product (usage stage), the consumer disposes of the used bioplastic product in the region and the disposed of bioplastic product is degraded in the environment (soil, seawater, and the like) of the region (disposal and degradation stage), a manufacturer in the region extracts vegetable oil from plants grown in the environment containing the degradation product generated during degradation (extraction stage), and a manufacturer in the region again produces a bioplastic product from the extracted vegetable oil (production stage).

The above-mentioned life cycle is the life cycle of an ecological system in a certain region which includes an environment in which the bioplastic product is introduced and the people involved with the bioplastic product, and is a life cycle relating to a bioplastic product. The life cycle assessment processing device 100 in the second embodiment evaluates the emission amount by taking into account the life cycle of the ecological system. Further, the life cycle assessment processing device 100 in the second embodiment corrects the emission amount by using an index indicating the quality of life of the people included in the ecological system.

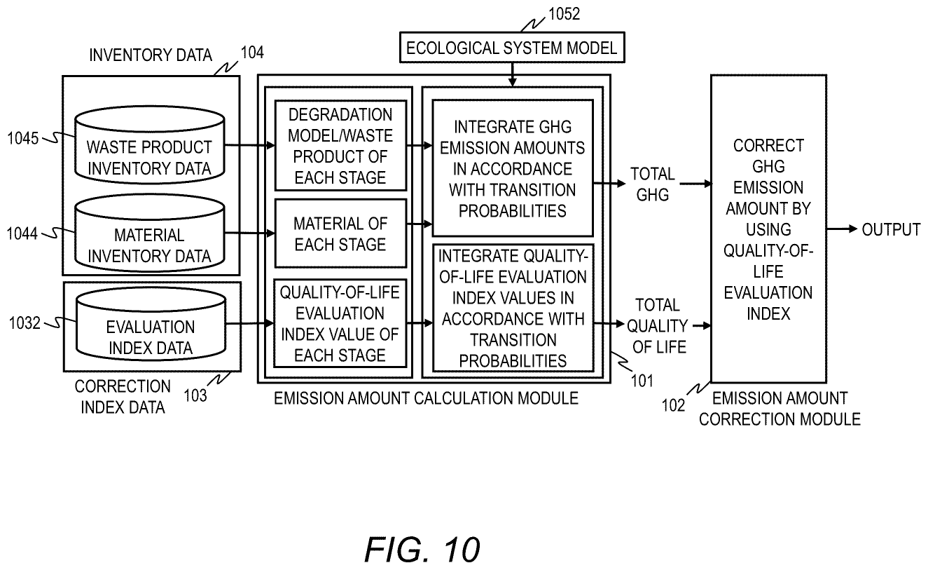

FIG. 10 is an explanatory diagram for illustrating an example of processing executed by the emission amount calculation module 101 and the emission amount correction module 102. The life cycle assessment processing device 100 in the second embodiment calculates, when a bioplastic product is produced, used, and disposed of in a certain region, the emission amount relating to the bioplastic product which takes into account the life cycle of the people and the environment of the region.

The emission amount calculation module 101 acquires an ecological system model 1052 corresponding to a bioplastic product as a life cycle model 105. The ecological system model 1052 indicates a plurality of stages of the ecological system relating to the bioplastic product and transition probabilities between the stages. Further, the ecological system model 1052 indicates the items which are used or produced at each stage. The item used in each stage may or may not be a biological product.

In at least one stage of the ecological system model 1052, a bioplastic product corresponding to the ecological system model 1052 is produced and/or used, and is degraded. In other words, the impact on the state of the ecological system from the introduction (degradation) of the evaluation-target bioplastic product into the environment included in the ecological system is reflected in the ecological system model 1052. Further, in each stage of the ecological system model 1052, items (materials or degradation products) different from the bioplastic product may be used or produced.

The ecological system model 1052 is a model for estimating a dynamic change of the states relating to the evaluation target product (the stages relating to the evaluation target product) of the ecological system into which the bioplastic product being an example of the evaluation target product is introduced.

The inventory data 104 includes, for example, material inventory data 1044 and waste product inventory data 1045. The material inventory data 1044 indicates the emission amount in the production process and/or distribution process of the materials used in the stages included in the ecological system model 1052. The waste product inventory data 1045 indicates a degradation model indicating a degradation product produced when a waste product is disposed of and degraded, and an emission amount produced by the production of the degradation product. That is, the waste products defined in the waste product inventory data 1045 are all biodegradable products, including bioplastic products.

The correction index data 103 includes evaluation index data 1032. The evaluation index data 1032 indicates an evaluation index for evaluating the quality of life of the people included in the ecological system at each stage included in the ecological system model 1052.

The emission amount calculation module 101 identifies the emission amount corresponding to the material, the emission amount corresponding to the degradation product corresponding to the waste product, and the quality-of-life evaluation index value of each stage included in the ecological system model 1052 from the material inventory data 1044, the waste product inventory data 1045, and the evaluation index data 1032, respectively.

The emission amount calculation module 101 causes the stages to transition in accordance with the transition probabilities indicated by the ecological system model 1052, and integrates the emission amounts and the quality-of-life evaluation index values for each stage. The emission amount calculation module 101 outputs the integrated emission amount (total emission amount) and the integrated quality-of-life index value (total quality of life) to the emission amount correction module 102. The total emission amount indicates the total of the emission amounts relating to the evaluation target product which takes into account the life cycle when the evaluation target product is introduced into the ecological system at any stage. The total quality of life indicates the total of the quality-of-life index values of (included in) the ecological system which takes into account the life cycle when the evaluation target product is introduced into the ecological system at any stage. The emission amount correction module 102 corrects the total emission amount by using the total quality of life, and outputs the corrected emission amount.

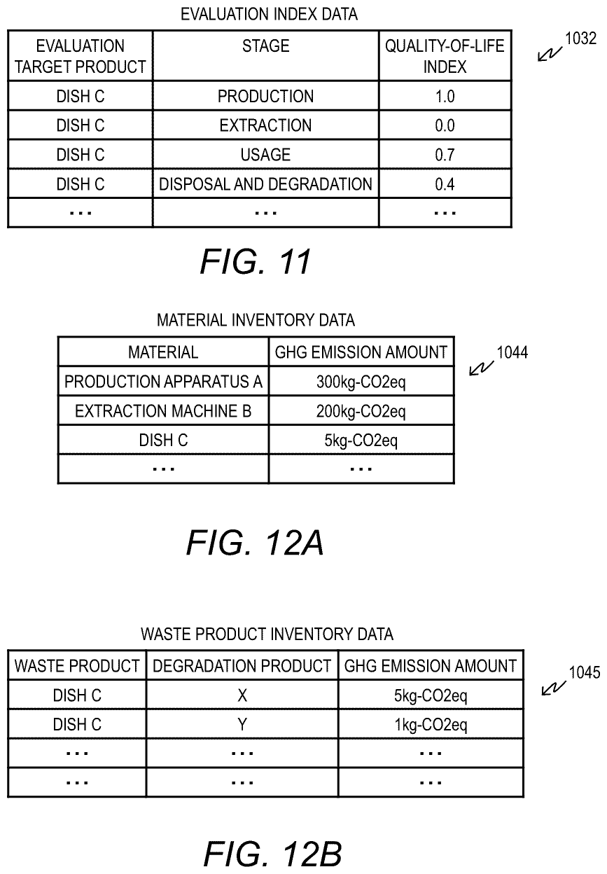

FIG. 11 is a table for showing a data configuration example of the evaluation index data 1032. The evaluation index data 1032 holds a quality-of-life index value for each combination of an evaluation target product and a stage. The evaluation index data 1032 is not required to include the evaluation target product field, that is, the quality-of-life index value may be set to be the same value when the stage is the same regardless of the evaluation target product.

FIG. 12A is a table for showing a data configuration example of the material inventory data 1044. The material inventory data 1044 indicates the emission amount (emission amount in the production process and/or distribution process of the material) corresponding to each predetermined unit amount (for example, one) of the material.

FIG. 12B is a table for showing a data configuration example of the waste product inventory data 1045. The waste product inventory data 1045 indicates the degradation product (degradation model) produced when a waste product is disposed of, and the emission amount produced by the production of the degradation product when the predetermined unit amount of the waste product is disposed of.

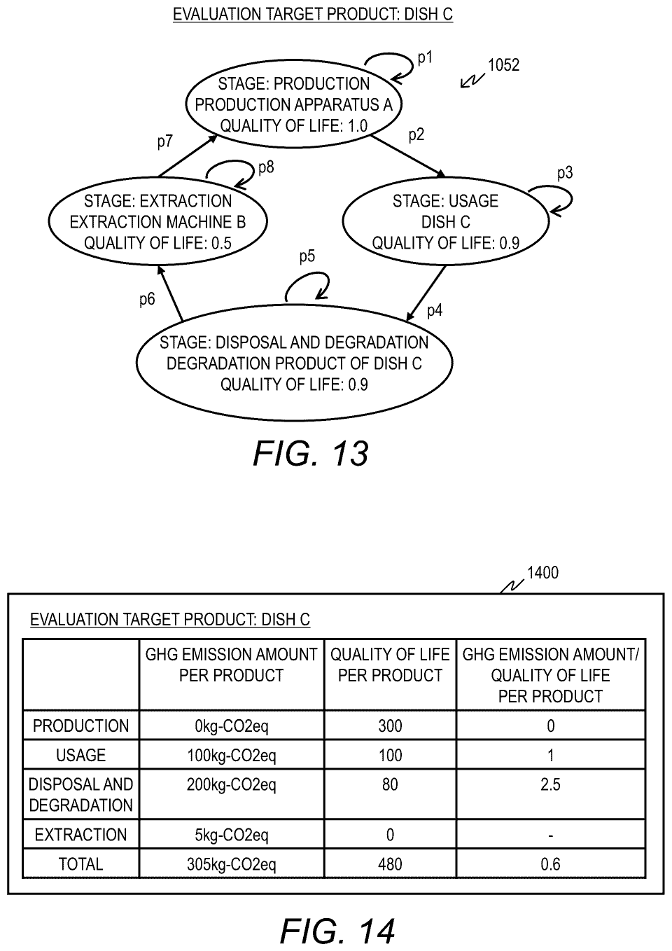

FIG. 13 is an explanatory diagram for illustrating an example of the ecological system model 1052. In FIG. 13, there is illustrated an example of an ecological system model 1052 (Markov chain model) when the bioplastic product which is the evaluation target product is a “dish C.”

The ecological system model 1052 defines a plurality of stages (“production” of “dish C,” “extraction” of vegetable oil for producing “dish C,” “usage” of “dish C,” and “disposal and degradation” of “dish C”) and the materials used and/or degradation products produced at each stage. The ecological system model 1052 may include stages in which no materials are used and no degradation products are produced.

Materials which are different from the evaluation target product used in each stage of the ecological system model 1052 and which are included in the material inventory data 1044 (“production apparatus A” and “extraction machine B” in the example of FIG. 13) are examples of a “used item.” Further, the degradation product produced at each stage (the “degradation product of dish C” in the example of FIG. 13) is an example of a “produced substance” at that stage. It should be noted that the ecological system model 1052 illustrated in the example of FIG. 13 includes a stage in which only “dish C,” which is the evaluation target product, is disposed of as a waste product and degraded, but the ecological system model 1052 may further include a stage in which materials other than the evaluation target product are disposed of as a waste product and degraded.

In the example of FIG. 13, the quality-of-life index value of each stage is also defined in the ecological system model 1052. However, at the stage at which the emission amount calculation module 101 acquires the ecological system model 1052, the quality-of-life index value is not defined, and the emission amount calculation module 101 assigns the quality-of-life index value of each stage to the ecological system model 1052 based on the evaluation index data 1032. The quality-of-life index value may also be defined in advance in the ecological system model 1052 acquired by the emission amount calculation module 101. In this case, the emission amount calculation module 101 is not required to acquire the evaluation index data 1032, and the processing of assigning the quality-of-life index value can also be omitted.

In the ecological system model 1052, transitions to the same stage may be defined. In addition, there may be cases in which transitions between specific stages are not defined in the ecological system model 1052.

In the ecological system model 1052, p1 to p9 attached to respective arrows each indicate the transition probability of the stage of the ecological system transitioning to the stage indicated by the end point of the arrow when a predetermined unit period (for example, one month) has elapsed since the stage of the ecological system transitioned to the stage indicated by the starting point of the arrow.

For example, when the stage is “extraction,” a unit amount (which is the same as the unit amount corresponding to the emission amount indicated by the material inventory data 1044) of the extraction machine B is used in the predetermined unit period. Under this condition, when the predetermined unit period has elapsed since the stage transitioned to “extraction,” the stage transitions to “production” at a transition probability p7, and transitions to “extraction” at a transition probability p8.

The life cycle assessment processing in the second embodiment is the same as the life cycle assessment processing in the first embodiment, except for the point that the ecological system model 1052 corresponding to the evaluation target product is used instead of the clinical condition transition model 1051 corresponding to the indication and the evaluation-target pharmaceutical product, the point that the material inventory data 1044 and the waste product inventory data 1045 are used instead of the pharmaceutical product inventory data 1041, the medical material inventory data 1042, and the metabolite inventory data 1043, the point that the evaluation index data 1032 is used instead of the QALY data 1031, and the point that the simulation termination condition is that a predetermined unit period has elapsed.

FIG. 14 is a diagram for illustrating a screen configuration example of the assessment processing result display screen. An assessment processing result display screen 1400 displays, for example, information indicating the designated evaluation target product (“drug A”). Further, the assessment processing result display screen 1400 displays information indicating the emission amount per evaluation target product (unit amount), the quality-of-life index value per evaluation target product (unit amount), and the corrected emission amount (emission amount/quality-of-life index value) per evaluation target product (unit amount) for each stage as a total value. The assessment processing result display screen 1400 of FIG. 14 allows the user of the life cycle assessment processing device 100 to recognize the emission amount, the quality-of-life index value, and the corrected emission amount at each stage.

As described above, the life cycle assessment processing device 100 in the second embodiment can calculate the emission amount in a life cycle of an ecological system into which a bioplastic product (biodegradable product) is introduced, and further can use a quality-of-life index of the ecological system to correct the emission amount.

This invention is not limited to the above-described embodiments but includes various modifications. The above-described embodiments are explained in details for better understanding of this invention and are not limited to those including all the configurations described above. A part of the configuration of one embodiment may be replaced with that of another embodiment; the configuration of one embodiment may be incorporated to the configuration of another embodiment. A part of the configuration of each embodiment may be added, deleted, or replaced by that of a different configuration.

The above-described configurations, functions, and processors, for all or a part of them, may be implemented by hardware: for example, by designing an integrated circuit. The above-described configurations and functions may be implemented by software, which means that a processor interprets and executes programs providing the functions. The information of programs, tables, and files to implement the functions may be stored in a storage device such as a memory, a hard disk drive, or an SSD (Solid State Drive), or a storage medium such as an IC card, or an SD card.

The drawings show control lines and information lines as considered necessary for explanations but do not show all control lines or information lines in the products. It can be considered that almost of all components are actually interconnected.

What is claimed is:

1. An information processing device, comprising:
a processor; and
a memory,
wherein the memory holds:
a life cycle model indicating information for estimating a dynamic change of states relating to an evaluation target product of one of a biological system or an ecological system into which the evaluation target product is introduced, and the evaluation target product and a used item other than the evaluation target product which are used in each of the states;
environmental load information indicating an environmental load in a production process and a distribution process of each of the evaluation target product and the used item; and
index information indicating an index value relating to, for each of the states, a quality of life of the one of the biological system or the ecological system affected by the evaluation target product, and
wherein the processor is configured to:
estimate the dynamic change of the states based on the life cycle model and identify the evaluation target product and the used item used in each of the states which stays in the estimated dynamic change;
calculate the environmental load in an entire period of the estimated dynamic change based on the environmental load corresponding to the identified evaluation target product and used item in the environmental load information;
calculate the index value in the entire period of the estimated dynamic change based on the index value corresponding to each of the states which stays in the estimated dynamic change in the index information;
correct the calculated environmental load based on the calculated index value; and
generate data for displaying the corrected environmental load.

2. The information processing device according to claim 1,
wherein the processor is configured to correct the calculated environmental load by substituting the calculated environmental load and the calculated index value into a predetermined function, and
wherein the predetermined function is a function which outputs a larger value when the calculated environmental load is larger, and outputs a smaller value when the calculated index value is larger.

3. The information processing device according to claim 2, wherein the predetermined function is a function which divides the calculated environmental load by the calculated index value.

4. The information processing device according to claim 1,
wherein the evaluation target product is an evaluation-target pharmaceutical product,
wherein the one of the biological system or the ecological system into which the evaluation target product is introduced is a patient into which the evaluation-target pharmaceutical product is introduced,
wherein each of the states indicates a clinical condition of the patient relating to an indication of the evaluation-target pharmaceutical product,
wherein the used item includes a pharmaceutical product other than the evaluation-target pharmaceutical product and a medical material which are used in each clinical condition, and
wherein the index value is a quality-adjusted life year of the patient.

5. The information processing device according to claim 4,
wherein the environmental load information indicates a metabolite produced when each of the evaluation-target pharmaceutical product and the pharmaceutical product included in the used item is introduced into the patient, and the environmental load produced by the metabolite, and
wherein the processor is configured to:
identify the metabolite corresponding to each of the identified evaluation target product and used item by referring to the environmental load information; and
calculate the environmental load in the entire period of the dynamic change based on the environmental load corresponding to the identified evaluation target product, used item, and metabolite in the environmental load information.

6. The information processing device according to claim 1,
wherein the evaluation target product is an evaluation-target biological product,
wherein the one of the biological system or the ecological system into which the evaluation target product is introduced is a first ecological system including an environment into which the evaluation-target biological product is introduced to be degraded,
wherein each of the states indicates a stage of the first ecological system relating to the evaluation-target biological product,
wherein the used item includes a material other than the evaluation-target biological product which is used in the stage, and
wherein the index value is an index value of the quality of life of a person included in the first ecological system.

7. The information processing device according to claim 6,
wherein the stage includes a degradation stage in which the evaluation-target biological product is introduced into the environment and is degraded,
wherein the environmental load information indicates a degradation product produced when the evaluation-target biological product is introduced into the environment and is degraded, and the environmental load produced by the degradation product, and
wherein the processor is configured to:
identify the degradation product produced in the degradation stage by referring to the environmental load information when the first ecosystem stays in the degradation stage during the estimated dynamic change; and
calculate the environmental load in the entire period of the dynamic change based on the environmental load corresponding to the identified evaluation target product, used item, and degradation product in the environmental load information.

8. The information processing device according to claim 1, wherein the processor is configured to:
calculate, for each of the states, the environmental load in the entire period of the estimated dynamic change based on the environmental load corresponding to the identified evaluation target product and used item in the environmental load information;
calculate, for each of the states, the index value in the entire period of the estimated dynamic change based on the index value corresponding to each of the states which stays in the estimated dynamic change in the index information;
correct, for each of the states, the calculated environmental load based on the calculated index value; and
generate data for displaying the calculated environmental load of the each of the states, the calculated index value of the each of the states, and the corrected environmental load of the each of the states.

9. An information processing method by an information processing device,
the information processing device comprising a processor and a memory,
the memory holding:
a life cycle model indicating information for estimating a dynamic change of states relating to an evaluation target product of one of a biological system or an ecological system into which the evaluation target product is introduced, and the evaluation target product and a used item other than the evaluation target product which are used in each of the states;
environmental load information indicating an environmental load in a production process and a distribution process of each of the evaluation target product and the used item; and
index information indicating an index value relating to, for each of the states, a quality of life of the one of the biological system or the ecological system affected by the evaluation target product,
the information processing method comprising:
estimating, by the processor, the dynamic change of the states based on the life cycle model and identify, by the processor, the evaluation target product and the used item used in each of the states which stays in the estimated dynamic change;
calculating, by the processor, the environmental load in an entire period of the estimated dynamic change based on the environmental load corresponding to the identified evaluation target product and used item in the environmental load information;
calculating, by the processor, the index value in the entire period of the estimated dynamic change based on the index value corresponding to each of the states which stays in the estimated dynamic change in the index information;
correcting, by the processor, the calculated environmental load based on the calculated index value; and
generating, by the processor, data for displaying the corrected environmental load.
